(19) United States
(12) Patent Application Publication
(10) Pub. No.: US 2009/0264507 A1
(43) Pub. Date: Oct. 22, 2009

(54) METHOD OF DIAGNOSING GUM DISEASE-WOUND HEALING USING SINGLE NUCLEOTIDE POLYMORPHISM PROFILES

(75) Inventors: Ichiro Nishimura, Santa Monica, CA (US); Audrey Lin, Los Angeles, CA (US); Neal Garret, Moorpark, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, Oakland, CA (US)

(21) Appl. No.: 11/817,339

(22) PCT Filed: Mar. 10, 2006
(86) PCT No.: PCT/US06/08678

§ 371 Date: Nov. 25, 2008

Related U.S. Application Data
(60) Provisional application No. 60/660,860, filed on Mar. 10, 2005.

(51) Int. Cl. A61K 31/7088; C12Q 1/68; C07H 21/04; C07K 16/18; A61P 1/02

(52) U.S. Cl. 514/44 R; 435/6; 536/24.33; 530/387.5

(57) ABSTRACT

The present invention provides methods for diagnosing and providing a prognosis for wound healing in a patient by characterizing and analyzing the single nucleotide polymorphism (SNP) of a wound healing associated gene wit3.0. In particular, the invention provides a method of diagnosis and prognosis of oral disease-wound healing, including gingival periodontitis and residual alveolar bone resorption, by characterizing and analyzing the wound inducible transcript-3.0 (wit3.0)SNP pattern in a sample of a patient with and without susceptibility for the disease.

### CROSS-REFERENCES TO RELATED APPLICATIONS

The present application claims the benefit of U.S. Ser. No. 60/660,860, filed Mar. 10, 2005, herein incorporated by reference in its entirety.

### STATEMENT AS TO RIGHTS TO INVENTIONS MADE UNDER FEDERALLY SPONSORED RESEARCH AND DEVELOPMENT

Not applicable.

### FIELD OF THE INVENTION

The present invention relates generally to methods for screening and diagnosis of gum disease and more specifically to characterizing and analyzing single nucleotide polymorphism (SNP) and haplotype patterns of genes associated with tissue wound healing, in particular, gum disease, including periodontitis and residual alveolar bone resorption.

### BACKGROUND

Tissue wound healing to restore the structural integrity of the damaged tissue involves a complex set of cellular and molecular events (e.g. proliferation of cells and healthy granulation formation). Failure of these cellular and molecular events, as well as other factors which impair the formation of healthy granulation or inhibit the inflammatory process (e.g. diabetes, vascular disease, immunosuppressant, etc.) produces abnormal wound healing and scarring.

Single nucleotide polymorphisms, or SNPs, are small genetic changes, or variations, that can occur within a any DNA sequence. On average, SNPs occur in the human population more than 1 percent of the time. A haplotype represents an allele, which may have one or more SNP combinations. Diagnostic tools utilizing SNPs and haplotypes provide a means for studying the genetics of diseases, not only for the purpose of detection but also for preventative and curative treatments of disease. In particular, late stages of gum disease is difficult and costly to treat (e.g. dental implant). Hence, an improved and more cost-effective method to detect gum disease before its onset or to improve treatment of existing gum disease is advantageous.

Wound inducible transcript-3.0 (wit3.0) is a gene differentially expressed in wounded oral mucosa cells. Characterization of the wit3.0 gene and its expression in edentulous oral mucosa undergoing tooth extraction wound healing is described in U.S. Ser. No. 10/170,786; WO 2002/100250; Sukotjo et al. (2002); and Sukotjo et al. (2003), all of which are hereby incorporated by reference in their entirety.

Thus, a need exists for a genetic test that will screen for a gum disease associated gene, such as wit3.0, and its associated SNPs, SNP patterns, haplotypes, or haplotype combinations. Wit3.0 SNP and haplotype profiles of a particular gum disease will assist in the prognosis for those patients with the disease or to prevent the onset of the disease in those who may or may not as yet be susceptible.

We have performed an in vitro study employing collagen gel embedded fibroblasts. The data showed that the over-expression of human wit3.0 with SNP1 and SNP1&2, both of which substitute amino-acid(s), significantly increased the gel contraction rate as compared to wild-type wit3.0. The data has been interpreted to indicate that patients carrying, for example, a wit3.0 SNP1 genotype, exhibit over contraction of tooth extraction wounds, resulting in an excessive alveolar bone resorption under the wound. This condition is consistent with clinical cases of highly resorbed edentulous jaw. These patients often experience difficulties for adapting dentures that are not stable, and for dental implant placement due to the lack of bone height and volume. We believe that a wit3.0 genotype test can predict the patients' poor prognosis of tooth extraction wound healing. This test can be used to provide a prognosis for tooth extraction healing and, for example, to advise dentists and patients to: (1) use other options than tooth extraction; and (2) place implant(s) immediately before losing the alveolar bone.

### SUMMARY OF THE INVENTION

The present invention is based on a method for screening a DNA sample for a plurality of target sequences having at least one known nucleotide variant (SNP 1 or SNP 2, or any combination thereof, including other wit3.0 SNPs). The target sequence is characterized by first contacting a sample containing the known target sequences with a detectable probe that specifically hybridizes to the target sequence of interest, and then subsequently detecting the detectable probe which is hybridized to the target sequence of interest.

In one embodiment of the invention, at least two different probes are utilized, each probe specifically hybridizing to a different target sequence. For example, the target sequence is wound inducible transcript 3.0 (wit3.0), and has at least one known variant, or a single nucleotide polymorphism (SNP), wherein the SNP is associated with a genetic disease. The genetic disease can include gingival periodontitis, residual alveolar bone resorption and other gum and oral related diseases. Alternatively, the SNP is associated with the efficacy of a drug for treatment of the disease.

In one embodiment, the present invention provides a method of providing a prognosis for oral and skin wound healing, e.g., tooth extraction wound healing and/or residual ridge alveolar bone resorption, by detecting wit3.0 SNPs or haplotypes, or combinations thereof.

In another embodiment of the invention, there is provided a method of treating a patient having gum disease by administering a therapeutically effective amount of an agent that stimulates wit3.0 expression or activity in a patient thereof. The agent can be, for example, wound inducible transcript (wit3.0), alone, or in a vector. A therapeutically effective amount is from about 10-200 ug or about 50-200 ug of wit3.0 in a buffer solution, for example, a tris-EDTA solution.

In another embodiment of the invention, there is provided a method of enhancing wound healing in the gum and skin by administering a therapeutically effective amount of an agent that stimulates wit3.0 expression or activity in a patient thereof. The agent can be, for example, wound inducible transcript (wit3.0), e.g., in a vector for therapeutic gene transfer to a cell, or in a nano-capsule for expression of Wit protein. The agent can also be a small molecule, peptide, antisense reagent, etc. that stimulates wit3.0 or Wit expression or activity includes increased transcription, post transcriptional processing, translation, post-translational processing, cellular localization (e.g, organelle, cytoplasm, nucleus, cell surface), and RNA and protein stability, etc.

The present invention also provides methods of assaying for compounds that provide enhanced wound healing in gum and skin, by assaying for agents that stimulate wit3.0 or Wit expression or activity. Stimulation of Wit expression or activity includes increased transcription, post transcriptional processing, translation, post-translational processing, cellular localization (e.g, organelle, cytoplasm, nucleus, cell surface), and RNA and protein stability, etc.

### BRIEF DESCRIPTION OF THE DRAWINGS

FIG. 1 shows two photographs of a post tooth extraction wound site. FIG. 1A shows a sutured wound site after tooth extraction. FIG. 1B shows the closed wound site within 1 week. Tooth extraction wound (left) heals with a rapid wound closure (right) within 1 week. Wit3.0 was isolated from the oral wound tissue.

FIG. 2 is a photograph of wound healing in mice with and without wit3.0 treatment. Identical skin wounds on the back skin of a mouse were treated with wit3.0 (left) and vehicle (right). The wit3.0 treated skin wound closed rapidly and tightly, which left no scarring.

FIG. 3 are two Confocal Laser Scanning Microscopic (CLSM) photographs of mouse skin wounds with (FIG. 3B) and without (FIG. 3B) wit3.0 treatment. Granulation tissue is indicated by (*) and double arrowheads; single arrowheads indicate hair follicles and sweat glands; and arrows indicate the wound margin between the wound edge and the granulation tissue. Collagen fibers were stained with Sirius Red. (A) Control wound showed sharp demarcation (arrows) between the wound edge and a large granulation tissue (*). Skin tissue contains hair follicles and sweat glands (arrowheads). The think and wavy collagen fibers stained red. The wound granulation tissue contains immature collagen fibers faintly stained red. (B) wit3.0 treated wound. The wound margins are pulled together (double-head arrow), flanking a small granulation tissue (*). The thick and ordered skin collagen fibers appeared to unite at the bottom of the wound, showing skin tissue regeneration.

FIG. 4 is a series of photographs of the gum line in rats treated with and without wit3.0. (Cont): Gingival tissue is firmly attached to the molar teeth. From the edge of molar teeth to the superior edge of gingival tissue is maintained (black double-head arrow), and the alveolar bone height is maintained as observed in micro-CT (white arrows). (Lig-induc. Perio): Placing a piece of suture (6.0) around the second molar at the level of gingival tissue induced periodontitis. After 10 days, inflammation in gingival tissue consistent of periodontitis was established. The height of inflamed gingival tissue in periodontitis was maintained (double-head arrow), while the underlying alveolar bone was significantly resorbed (white arrows). This should lead to the deepened gingival pocket. (Lig-induc.Perio, wit3.0 Tx): naked plasmid DNA vector containing the coding sequence of wit3.0 was applied in the gingival pocket. After 7 days, a significant tightening of gingival tissue was observed. As the result, the diseased gingival tissue was re-attached to the molar teeth and the reduction of pathological periodontal pocket appeared to be accomplished without a conventional periodontal surgery.

FIG. 5 is a series of diagrams showing the chromosomal location in both human and rat (FIG. 5A); the exon and intron structure (FIG. 5B); and the deduced peptide structure (FIG. 5C).

FIG. 6 shows the amino acid sequence for human wit3.0, including the haplotypes at amino acid residues 154 and 155.

FIG. 7 shows the nucleotide sequence for human wit3.0, including SNPs at nucleotides 739 and 740.

FIG. 8 shows the SNPs for human beta wit3.0.

### DETAILED DESCRIPTION OF THE INVENTION

### 1. Introduction

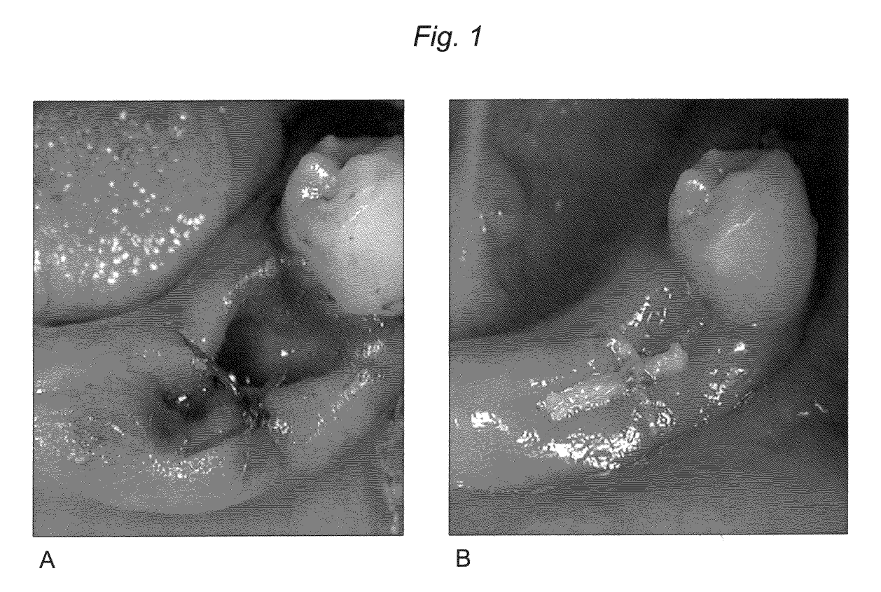

It is widely known that wound in the oral cavity heals better than skin. Oral wounds such as tooth extraction heal faster and leave less scarring (FIG. 1). We have recently isolated a unique gene from oral wound, named “wound inducible transcript-3.0” or wit3.0 (see published U.S. application Ser. No. 10/170,786; WO 2002/100250; Sukotjo et al., J. Dent. Res. 81(4):229-235 (2002); and Sukotjo et al., J. Biol Chem. 278(51):51527-51534 (2003), all of which are hereby incorporated by reference in their entirety). We have found that wit3.0 is synthesized only when injury is created, and wit3.0 helps fibroblasts to pull the surrounding tissue, which may be one of the biological mechanisms to close the wound.

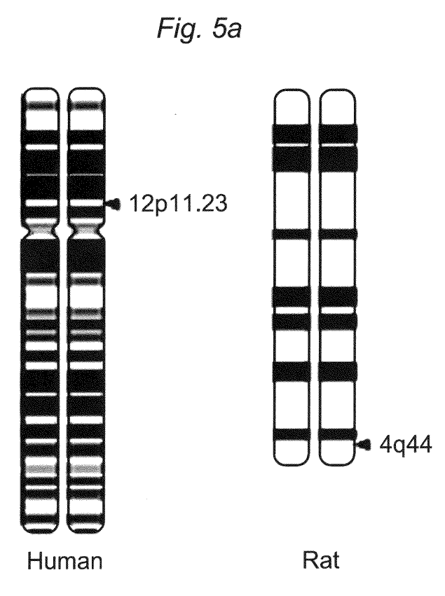

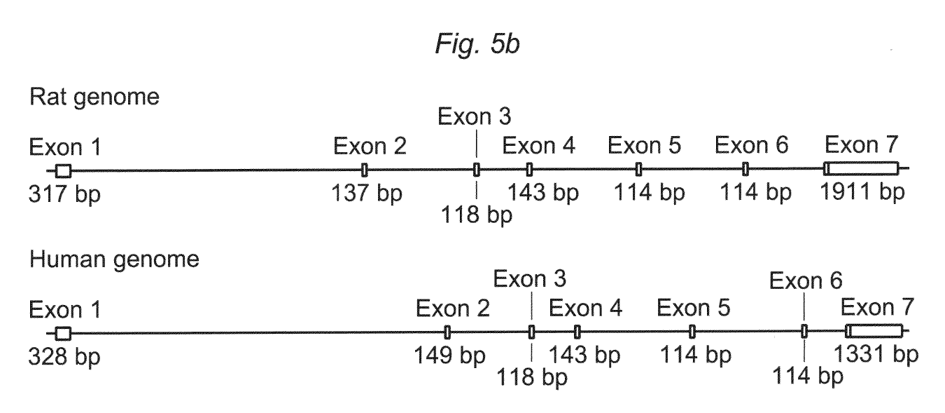

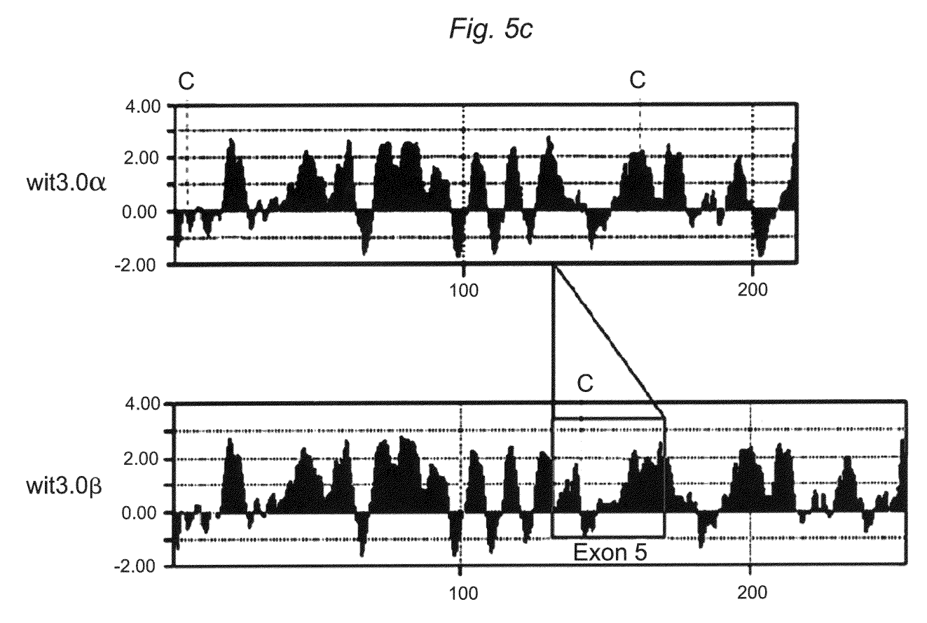

One embodiment of the invention provides prognostic and diagnostic uses for wit3.0 SNPs and haplotypes. There have been numerous successes identifying genes mutated in monogenic disorders using positional cloning. Linkage in family studies is being supplemented with locus-specific association studies in populations, enabling accurate chromosomal localization of the disease causing or susceptibility gene. Utilizing the genomic DNA sequence databases of humans and other animals recently available, we have identified the chromosomal location of wit3.0 to be 12p11.23 in the human and 4q44 in the rat (FIG. 5). The wit3.0 SNP genotyping may be valuable for the prognosis of tooth extraction wound healing and following residual ridge alveolar bone resorption. Tooth extraction creates extensive ablation wound not only in alveolar bone but also in the gingival soft tissue. The wound healing gingival has been shown expressing wit3.0 over 30 times higher than unwounded gingival, suggesting that the rapid soft tissue wound contraction at the tooth extraction site may be largely accomplished by wit3.0. The clinically important variations have been observed for the short-term and the long-term change in residual alveolar bone height and volume, which ultimately support conventional or implant-assisted dental prostheses. In some patients, the residual alveolar ridge is highly resorbed, no dental treatment can be done. If we have any methods for assessing the prognosis of residual alveolar bone resorption, it would be applied for all tooth extraction procedures (the single largest surgical treatment in dentistry, performed 191,803 cases annually per 1,000 patients in 2003 in the USA). The patient's wit3.0 SNP genotype indicating the poor prognosis for preserving the residual alveolar bone may be recommended for prophylactic treatments such retaining the root in the bone or placing dental implant.

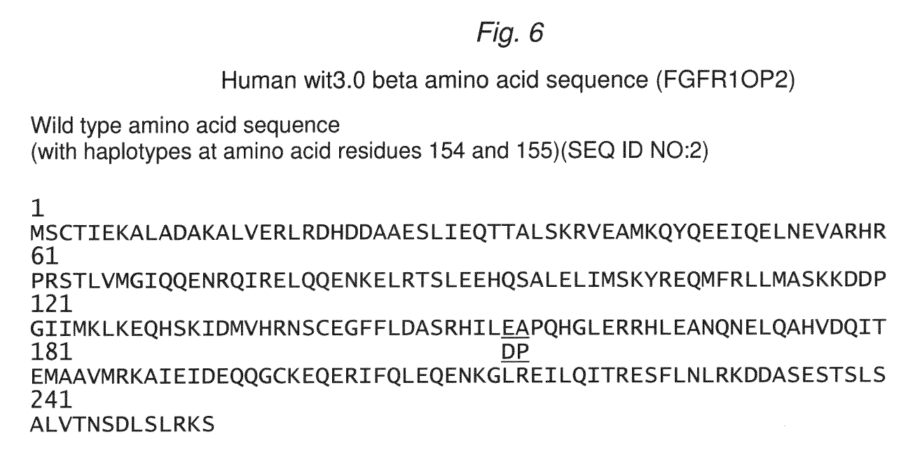

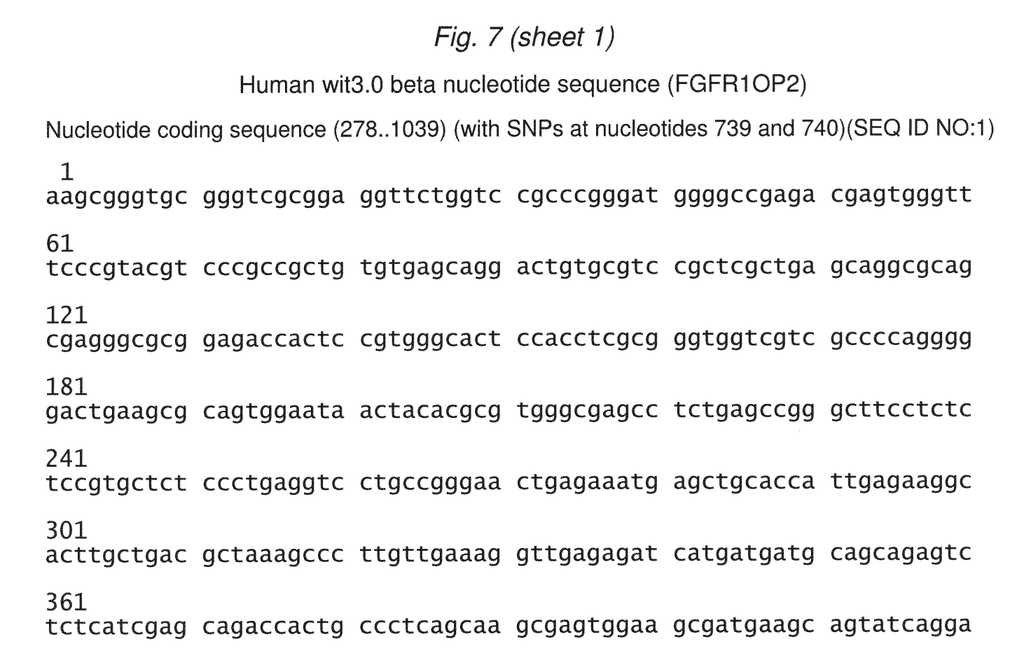

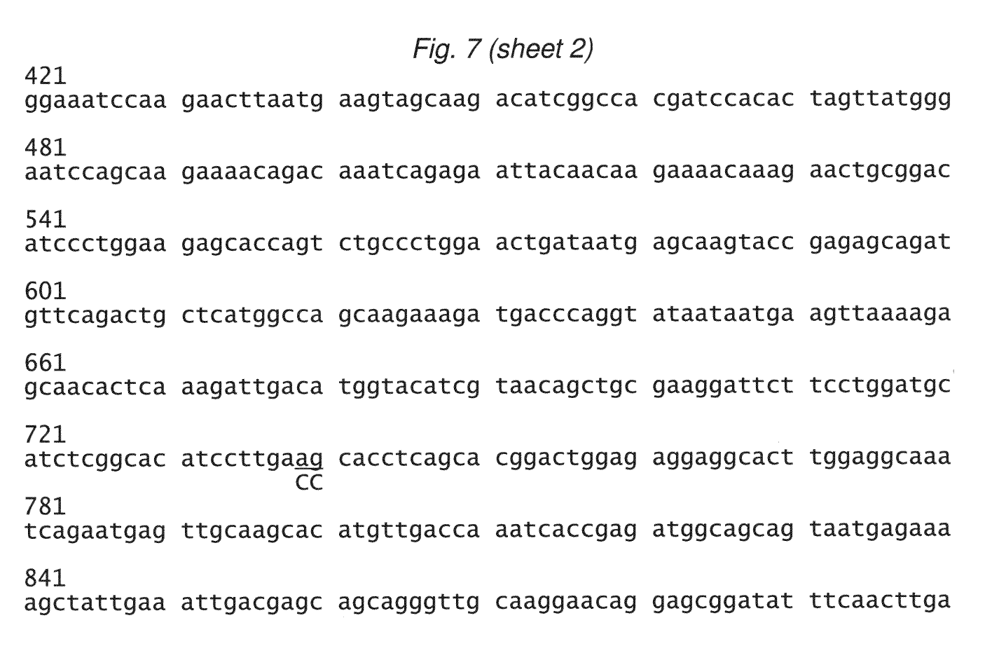

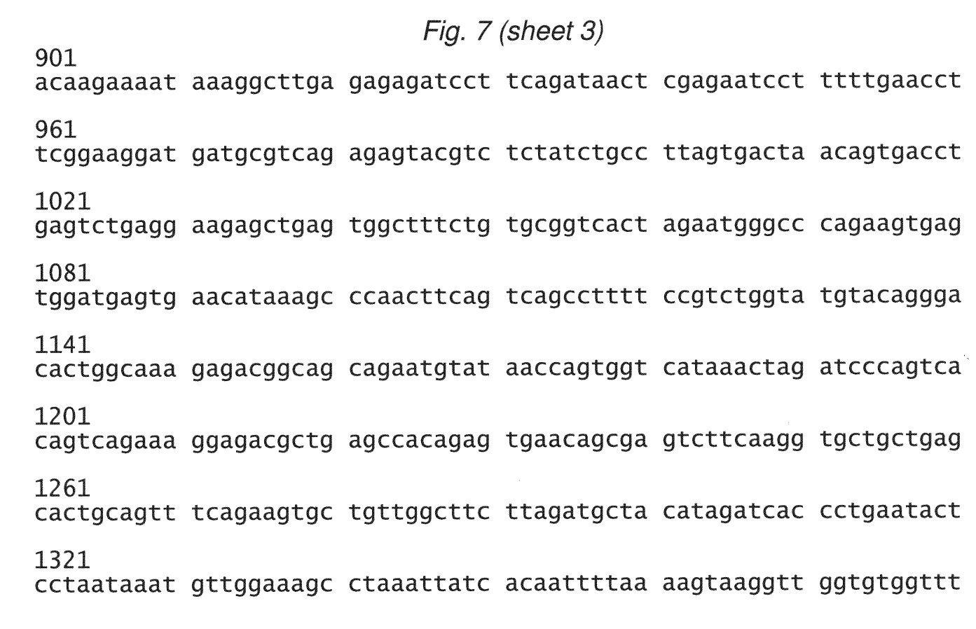

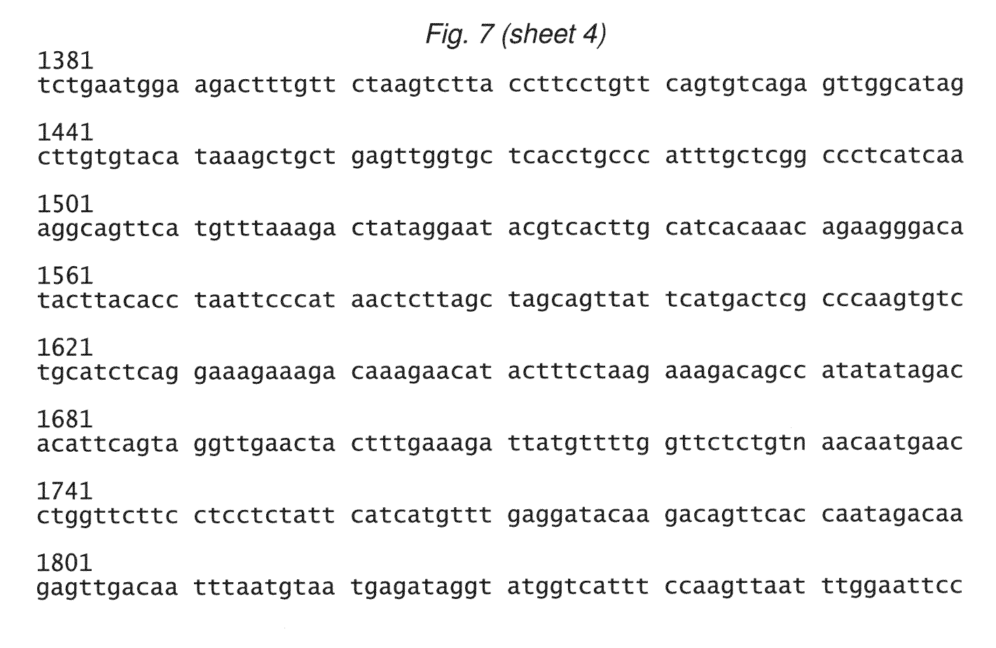

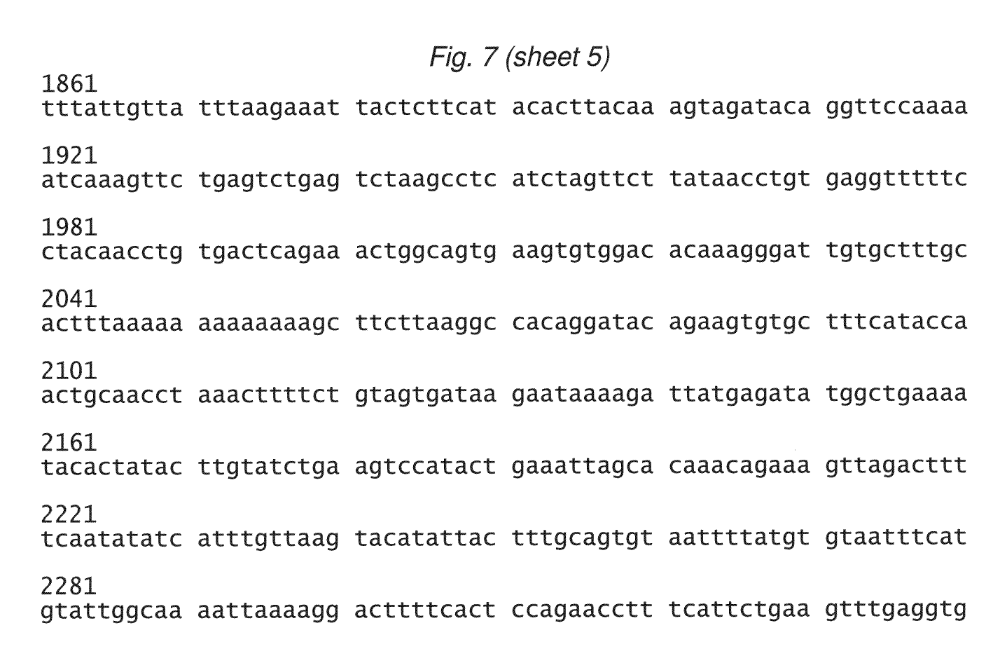

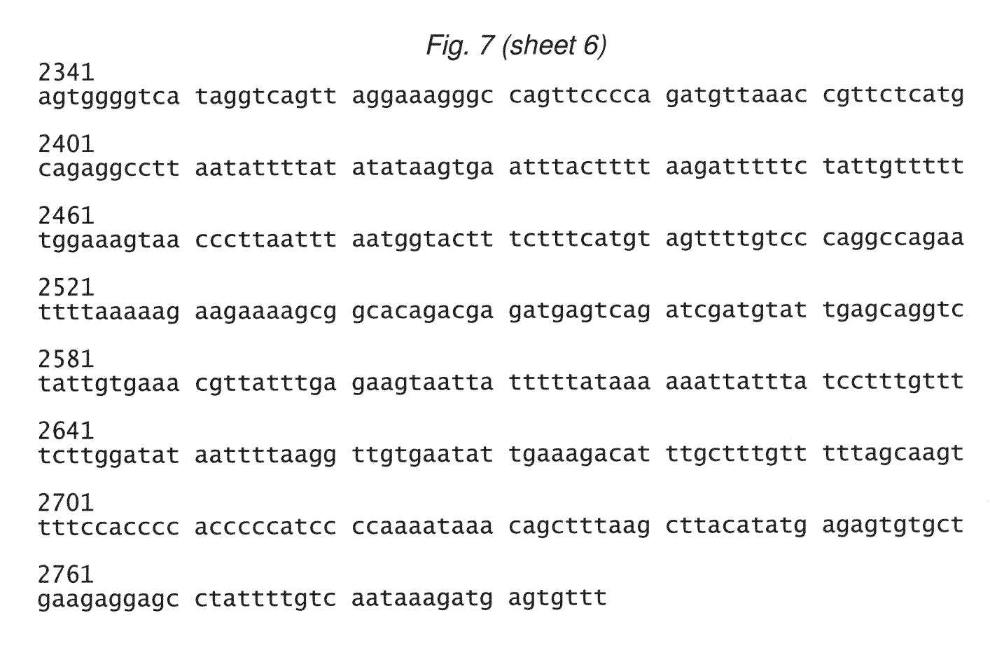

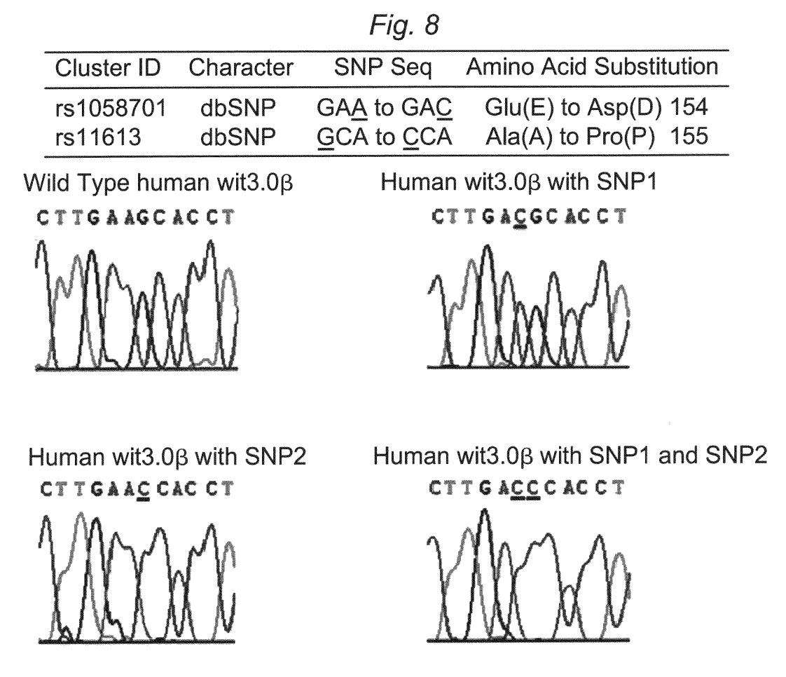

It is known that the degree of wound healing and scar formation significantly varies among different individuals. While fibroblasts derived from different origins are known to be phenotypically distinct, the genetic basis of individual variations on wound healing and scarring is far from understood. Single nucleotide polymorphism (SNP) mapping presents a new avenue for study of pathogenic genes, particularly of common diseases. Identification of SNPs in wit3.0 may enable better understanding of functional mechanisms. From the cluster ID located in human chromosome 12p11.23 (wit3.0 allele), the deposited SNP information was obtained from the NCBI web site. Out of 40 SNPs found in the wit3.0 allele, 2 SNPs were located in the coding sequence of exon 5, leading to the amino-acid substitution mutations. SNP 1 is at nucleotide 739 (A to C) of wit3.0 and SNP 2 is at nucleotide 740 (G to C) of wit3.0 (see FIG. 7 and NM_{—}026218). The amino acid substitution occurs at Glu(E) to Asp(D) at 154 (SNP1) and Ala(A) to Pro(P) at 155 (see FIG. 6 and NM_{—}026218). As the result, it is anticipated that 4 possible variations or haplotypes can exist in human wit3.0 coding sequence: GAAGCA=154Glu-155Ala (wild type), GACGCA=Asp-Ala (SNP 1), GAACCA=Glu-Pro (SNP 2), and GACCCA=Asp-Pro (SNP 1 and 2). Human wit3.0 genotype may thus carry 9 possible combinations of haplotypes (wt/wt; wt/SNP 1; wt/SNP 2; wt/SNP 1 and 2; SNP 1/SNP 1; SNP 1/SNP 2; SNP 1/SNP 1 and 2; SNP 2/SNP 2; and SNP 1 and 2/SNP1 and 2.

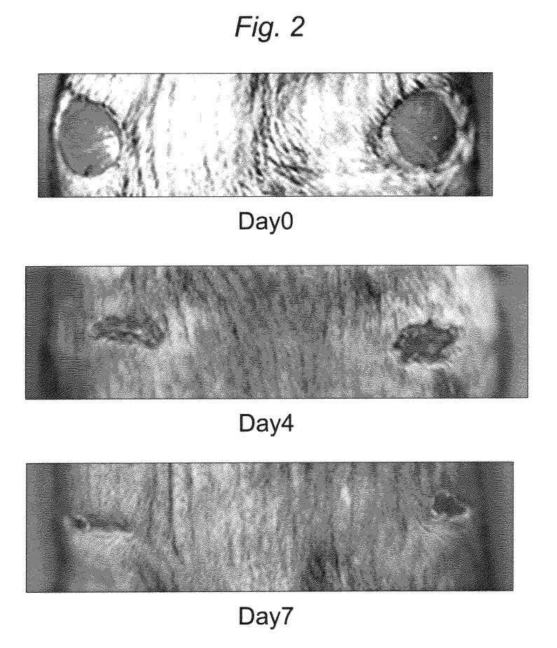

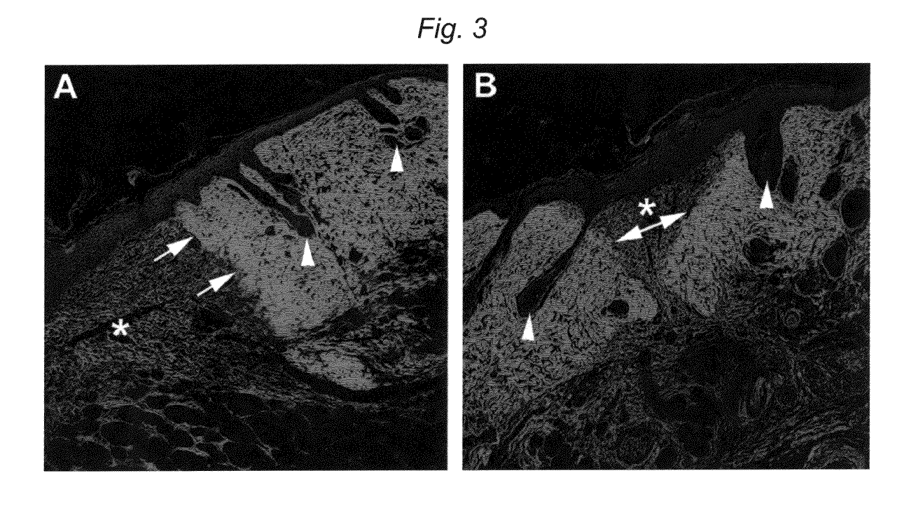

In another embodiment of the invention, because wit3.0 is less involved in skin wound, we demonstrate that if wit3.0 is more active in skin wound, it heals better. There are many ways to stimulate the wit3.0: (1) therapeutic gene transfer; (2) wit3.0 nano-capsule delivery; and (3) wit3.0 gene activation. Using a mouse skin model, we establish that the wit3.0 treated wound heals faster, stronger (without pulling apart), and better (less scarring). In example, two identical skin wounds (8 mm in diameter) were created on the back of mouse. The left side was treated with wit3.0 gene therapy and the right side was untreated control. The wit3.0 treated wound closed tightly even only after 4 days (FIG. 2). It was also noted that the wit3.0 treated wound closed in a linear fashion. This wound healing model is similar to intentional wound closure with suture placement. The suture placement greatly decreases the wound area and facilitates the healing. In order to avoid bacterial infection around the suture materials, doctors must remove sutures within 7 days. However, one week is not often enough to complete the wound closure and dehiscence or reopening of wound may occur. In particular, the maintenance of the skin suture has been challenging in veterinary medicine. The wit3.0 wound closure may change this wound suturing practice. Histological evaluation revealed that the control wound induced a large granulation tissue with faint collagen fibers that are different from surrounding normal skin. The granulation tissue also lacks appended skin structure such as hair and sweat glands. The healing outcomes of granulation tissue are tight skin due to unorganized collagen fibers, loss of hair, and scarring. The wit3.0 treated wound did not induce granulation tissue (FIG. 3). The wound edges were pulled together and in some areas the native collagen fibers started to regenerate.

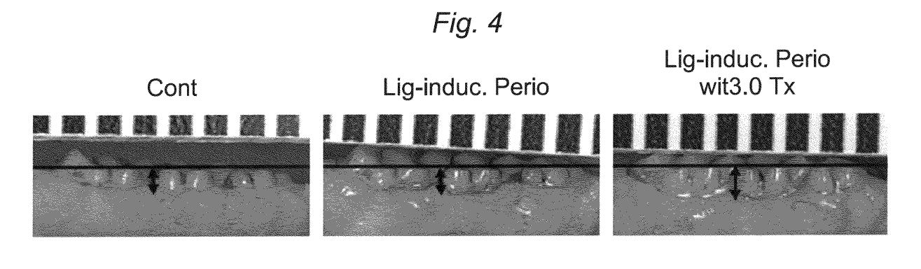

In another embodiment, wit3.0 gene therapy or activation can be used to treat periodontitis. Periodontitis is a common gum disease, which causes more tooth losses than decay in the adult population. Furthermore, because the life expectancy of the pet animals has recently extended, the aging and frail pets are suffering more tooth problems caused by periodontitis. Currently, the treatment of periodontitis is limited to tooth extraction or surgical removal of inflamed gum tissue (FIG. 4). In subjects treated with wit3.0 gene therapy, after 7 days, a significant tightening of gingival tissue was observed (FIG. 4). As the result, the diseased gingival tissue was re-attached to the molar teeth and the reduction of pathological periodontal pocket was accomplished without a conventional periodontal surgery. Pets can include, e.g., dogs, cats, rats, mice, guinea pigs, rabbits, and other domesticated animals such as horses, pigs, cows, donkeys, mules, etc.

In another embodiment, wit3.0 can be used in drug assays to identify activators and modulators of wit3.0 for therapeutic applications. Pharmacogenetic studies of wit3.0 SNPs and haplotypes can also be used to customize therapeutic treatments.

The present invention may be understood more readily by reference to the following detailed description of specific embodiments and the Examples included therein.

### 2. Definitions

“wit3.0” and “Wit” refer to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. The human protein and nucleic acid sequences are provided, for example, by Accession No. NM_{—}026218 and in FIG. 6 and 7. The rat protein and nucleic acid sequences for wit3.0 are provided, for example, by Accession Nos. NM_{—}201421, BC087696, AY426740 and AY426739. Truncated and alternatively spliced forms of these antigens are included in the definition.

The terms “overexpress,” “overexpression” or “overexpressed” interchangeably refer to a protein that is transcribed or translated at a detectably greater level, usually in a wounded oral cavity or skin cell (e.g., a fibroblast), in comparison to a normal cell. The term includes overexpression due to increased transcription, post transcriptional processing, translation, post-translational processing, cellular localization (e.g, organelle, cytoplasm, nucleus, cell surface), and RNA and protein stability, as compared to a normal cell. Overexpression can be detected using conventional techniques for detecting mRNA (i.e., RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a normal cell. In certain instances, overexpression is 1-fold, 2-fold, 3-fold, 4-fold or more higher levels of transcription or translation in comparison to a normal cell.

The term “improving wound healing” refers to enhancing, improving, hastening, ameliorating, etc. the healing of an oral or skin wound. Typically the cell type is a fibroblast (a cell ubiquitous in connective tissue that makes and secretes collagen ) or fibroblast-like cell (e.g., a connective tissue cell such as a bone cell or a fat cell), although additional cell types can also have improved wound healing, e.g., skeletal muscle cells, cardiac muscle cells, smooth muscle cells, and epithelial cells. This term also refers to the rate of healing (e.g., faster), the quality of healing (e.g., size, shape, orientation, color, and type of scar), as well as reduction in the pain and discomfort of healing due to faster rates and/or quality of healing. “Wound healing response” refers to the rate of healing and the quality of healing. Typically, improved wound healing is at least 10% better than a control as measured using rate or quality factors, preferably 20%, 30%, 50%, 75%, 100%, 500%, etc. better.

“Oral tissue” refers to any tissue in the mouth, pharynx, and throat, including the tongue, salivary glands, and gums.

“Oral wounds, diseases and conditions” include tooth extraction wound healing, residual ridge alveolar bone resorption, cancer, abcess, gingival periodontitis, vitamin deficiency, wounds from oral surgery, trauma, wounds from cancer surgery or treatment, such as radiation, viral disease, e.g., herpes infection, yeast or bacterial infection, e.g., thrush, leukoplakia, etc. “Skin wounds, diseases and conditions” include cancer, wounds from surgery, trauma, wounds from cancer surgery or treatment, such as radiation, viral disease, yeast or bacterial infection, etc.

As used herein “oligonucleotides” or “oligomers” refer to a nucleic acid sequence of approximately 7 nucleotides or greater in length, and up to as many as approximately 100 nucleotides in length, which can be used as a primer, probe or amplimer. Oligonucleotides are often between about 10 and about 50 nucleotides in length, more often between about 14 and about 35 nucleotides, very often between about 15 and about 30 nucleotides, and the terms oligonucleotides or oligomers can also refer to synthetic and/or non-naturally occurring nucleic acids (i.e., comprising nucleic acid analogues or modified backbone residues or linkages).

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al, J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of cancer antigens. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein having an amino acid sequence encoded by any of the polynucleotides of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors are agents that, e.g., inhibit expression of a polypeptide or polynucleotide of the invention or bind to, partially or totally block stimulation or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation or enzymatic activity, sensitize or up regulate the activity of a polypeptide or polynucleotide of the invention, e.g., agonists. Modulators include naturally occurring and synthetic ligands, antagonists, agonists, small chemical molecules and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a polypeptide or polynucleotide of the invention relative to the control is about 80%, optionally 50% or 25-1%. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” or “RNAi” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

The terms “peptidomimetic” and “mimetic” refer to a synthetic chemical compound that has substantially the same structural and functional characteristics of the polynucleotides, polypeptides, antagonists or agonists of the invention. Peptide analogs are commonly used in the pharmaceutical industry as non-peptide drugs with properties analogous to those of the template peptide. These types of non-peptide compound are termed “peptide mimetics” or “peptidomimetics” (Fauchere, Adv. Drug Res. 15:29 (1986); Veber and Freidinger TINS p. 392 (1985); and Evans et al., J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as a CCX CKR, but have one or more peptide linkages optionally replaced by a linkage selected from the group consisting of, e.g., —CH₂NH—, —CH₂S—, —CH₂—CH₂—, —CH═CH— (cis and trans), —COCH₂—, —CH(OH)CH₂—, and —CH₂SO—. The mimetic can be either entirely composed of synthetic, non-natural natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out the binding or enzymatic activities of a polypeptide or polynucleotide of the invention or inhibiting or increasing the enzymatic activity or expression of a polypeptide or polynucleotide of the invention.

“Determining the functional effect” refers to assaying for a compound that increases or decreases a parameter that is indirectly or directly under the influence of a polynucleotide or polypeptide of the invention, e.g., measuring physical and chemical or phenotypic effects. Such functional effects can be measured by any means known to those skilled in the art, e.g., changes in spectroscopic (e.g., fluorescence, absorbance, refractive index), hydrodynamic (e.g., shape), chromatographic, or solubility properties for the protein; measuring inducible markers or transcriptional activation of the protein; measuring binding activity or binding assays, e.g. binding to antibodies; measuring changes in ligand binding affinity; measurement of calcium influx; measurement of the accumulation of an enzymatic product of a polypeptide of the invention or depletion of an substrate; measurement of changes in protein levels of a polypeptide of the invention; measurement of RNA stability; G-protein binding; GPCR phosphorylation or dephosphorylation; signal transduction, e.g., receptor-ligand interactions, second messenger concentrations (e.g., cAMP, IP3, or intracellular Ca²⁺); identification of downstream or reporter gene expression (CAT, luciferase, β-gal, GFP and the like), e.g., via chemiluminescence, fluorescence, calorimetric reactions, antibody binding, inducible markers, and ligand binding assays.

Samples or assays comprising a nucleic acid or protein disclosed herein that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved when the activity value relative to the control is about 80%, preferably 50%, more preferably 25-0%. Activation is achieved when the activity value relative to the control (untreated with activators) is 110%, more preferably 150%, more preferably 200-500% (i.e., two to five fold higher relative to the control), more preferably 1000-3000% higher.

### 3. Diagnosis Using SNPs and Haplotypes

The present invention also provides methods of diagnosing or providing a prognosis for oral wound healing, gum disease, tooth extractions, and skin wound healing. Diagnosis involves determining the level of a polypeptide or polynucleotide of the invention in a patient and then comparing the level to a baseline or range. Typically, the baseline value is representative of a polypeptide or polynucleotide of the invention in a healthy person not suffering from the condition or disease state. Variation of levels of a polypeptide or polynucleotide of the invention from the baseline range (either up or down) indicates that the patient may have an altered prognosis or diagnosis, leading to differences in the way that the patient is treated medically. In some embodiments, the level of a polypeptide or polynucleotide of the invention are measured by taking a tissue sample from a patient and measuring the amount of a polypeptide or polynucleotide of the invention in the sample using any number of detection methods, such as those discussed herein.

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)). Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

Single nucleotide polymorphism (SNP) or haplotype analysis is useful for detecting differences between alleles of the polynucleotides (e.g., genes) of the invention. SNPs linked to genes encoding polypeptides of the invention are useful, for instance, for prognosis and diagnosis of gum disease, tooth disease, and wound healing, e.g., after tooth extraction, whose occurrence is linked to the gene sequences of the invention. For example, if an individual carries at least one SNP linked to a disease or condition-associated allele of the gene sequences of the invention, the individual is likely predisposed for one or more of those diseases or conditions. If the individual is homozygous for a disease or condition-linked SNP, the individual is particularly predisposed for occurrence of that disease. In some embodiments, the SNP associated with the gene sequences of the invention is located within 300,000; 200,000; 100,000; 75,000; 50,000; or 10,000 base pairs from the gene sequence.

Various real-time PCR methods can be used to detect SNPs and haplotypes, including, e.g., Taqman or molecular beacon-based assays (e.g., U.S. Pat. Nos. 5,210,015; 5,487,972; Tyagi et al., Nature Biotechnology 14:303 (1996); and PCT WO 95/13399) are useful to monitor for the presence of absence of a SNP. Additional SNP detection methods include, e.g., DNA sequencing, sequencing by hybridization, dot blotting, oligonucleotide array (DNA Chip) hybridization analysis, or are described in, e.g., U.S. Pat. No. 6,177,249; Landegren et al., Genome Research, 8:769-776 (1998); Botstein et al., Am J Human Genetics 32:314-331 (1980); Meyers et al., Methods in Enzymology 155:501-527 (1987); Keen et al., Trends in Genetics 7:5 (1991); Myers et al., Science 230:1242-1246 (1985); and Kwok et al., Genomics 23:138-144 (1994).

A variety of additional methods of specific DNA and RNA measurement using nucleic acid hybridization techniques are known to those of skill in the art (see, Sambrook, supra). Some methods involve an electrophoretic separation (e.g., Southern blot for detecting DNA, and Northern blot for detecting RNA), but measurement of DNA and RNA can also be carried out in the absence of electrophoretic separation (e.g., by dot blot). Southern blot of genomic DNA (e.g., from a human) can be used for screening for restriction fragment length polymorphism (RFLP) to detect the presence of a genetic disorder affecting a polypeptide of the invention.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands that bind to labeled antibodies, fluorophores, chemiluminescent agents, enzymes, and antibodies which can serve as specific binding pair members for a labeled ligand. An introduction to labels, labeling procedures and detection of labels is found in Polak and Van Noorden Introduction to Immunocytochemistry, 2nd ed., Springer Verlag, NY (1997); and in Haugland Handbook of Fluorescent Probes and Research Chemicals, a combined handbook and catalogue Published by Molecular Probes, Inc. (1996).

In general, a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

Most typically, the amount of RNA is measured by quantifying the amount of label fixed to the solid support by binding of the detection reagent. Typically, the presence of a modulator during incubation will increase or decrease the amount of label fixed to the solid support relative to a control incubation which does not comprise the modulator, or as compared to a baseline established for a particular reaction type. Means of detecting and quantifying labels are well known to those of skill in the art.

In preferred embodiments, the target nucleic acid or the probe is immobilized on a solid support. Solid supports suitable for use in the assays of the invention are known to those of skill in the art. As used herein, a solid support is a matrix of material in a substantially fixed arrangement.

A variety of automated solid-phase assay techniques are also appropriate. For instance, very large scale immobilized polymer arrays (VLSIPS™), available from Affymetrix, Inc. (Santa Clara, Calif.) can be used to detect changes in expression levels of a plurality of genes involved in the same regulatory pathways simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759.

Detection can be accomplished, for example, by using a labeled detection moiety that binds specifically to duplex nucleic acids (e.g., an antibody that is specific for RNA-DNA duplexes). One preferred example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) Proc. Nat'l Acad. Sci. USA 65:993-1000; Ballard (1982) Mol. Immunol. 19:793-799; Pisetsky and Caster (1982) Mol. Immunol. 19:645-650; Viscidi et al. (1988) J. Clin. Microbial 41:199-209; and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies that are commercially or publicly available. In addition to the art referenced above, general methods for producing polyclonal and monoclonal antibodies are known to those of skill in the art (see, e.g., Paul (3rd ed.) Fundamental Immunology Raven Press, Ltd., NY (1993); Coligan Current Protocols in Immunology Wiley/Greene, NY (1991); Harlow and Lane Antibodies: A Laboratory Manual Cold Spring Harbor Press, NY (1988); Stites et al. (eds.) Basic and Clinical Immunology (4th ed.) Lange Medical Publications, Los Altos, Calif., and references cited therein; Goding Monoclonal Antibodies: Principles and Practice (2d ed.) Academic Press, New York, NY, (1986); and Kohler and Milstein Nature 256: 495-497 (1975)). Other suitable techniques for antibody preparation include selection of libraries of recombinant antibodies in phage or similar vectors (see, Huse et al. Science 246:1275-1281 (1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through use of a nucleic acid amplification system that multiplies the target nucleic acid being detected. Examples of such systems include the polymerase chain reaction (PCR) system, in particular RT-PCR or real time PCR, and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferentially human cells from the cerebellum or the hippocampus, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

### 4. Kits

The invention provides also provides compositions, kits and integrated systems for practicing the assays described herein using polypeptides or polynucleotides of the invention, antibodies specific for polypeptides or polynucleotides of the invention, etc.

The invention provides assay compositions for use in solid phase assays; such compositions can include, for example, one or more polynucleotides or polypeptides of the invention immobilized on a solid support, and a labeling reagent. In each case, the assay compositions can also include additional reagents that are desirable for hybridization. Modulators of expression or activity of polynucleotides or polypeptides of the invention can also be included in the assay compositions.

The invention also provides kits for carrying out the diagnostic assays of the invention. The kits typically include a probe that comprises an antibody or nucleic acid sequence that specifically binds to polypeptides or polynucleotides of the invention, and a label for detecting the presence of the probe. The kits may include several polynucleotide sequences encoding polypeptides of the invention.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical images.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen are sampled to obtain light intensity readings for each position. Multiple pixels are processed in parallel to increase speed. The apparatus and methods of the invention are easily used for viewing any sample, e.g., by fluorescent or dark field microscopic techniques.

### 5. Screening for Modulators of Polypeptides and Polynucleotides of the Invention

Modulators of polypeptides or polynucleotides of the invention are useful for activating wit3.0 expression or activity. Administration of activators or other agents that modulate expression of the polynucleotides or polypeptides of the invention can be used to treat patients with skin or oral cavity wounds, tooth disease, and gum disease.

A. Screening Methods

A number of different screening protocols can be utilized to identify agents that modulate the level of expression or activity of polypeptides and polynucleotides of the invention in cells, particularly mammalian cells, and especially human cells. In general terms, the screening methods involve screening a plurality of agents to identify an agent that modulates the polypeptide activity by binding to a polypeptide of the invention, modulating inhibitor binding to the polypeptide or activating expression of the polypeptide or polynucleotide, for example.

### Binding Assays

Preliminary screens can be conducted by screening for agents capable of binding to a polypeptide of the invention, as at least some of the agents so identified are likely modulators of polypeptide activity. The binding assays usually involve contacting a polypeptide of the invention with one or more test agents and allowing sufficient time for the protein and test agents to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet and Yamamura, (1985) “Neurotransmitter, Hormone or Drug Receptor Binding Methods,” in Neurotransmitter Receptor Binding (Yamamura, H. I., et al., eds.), pp. 61-89. The protein utilized in such assays can be naturally expressed, cloned or synthesized.

Binding assays are also useful, e.g., for identifying endogenous proteins that interact with a polypeptide of the invention. For example, antibodies, receptors or other molecules that bind a polypeptide of the invention can be identified in binding assays.

### Expression Assays

Certain screening methods involve screening for a compound that up or down-regulates the expression of a polypeptide or polynucleotide of the invention. Such methods generally involve conducting cell-based assays in which test compounds are contacted with one or more cells expressing a polypeptide or polynucleotide of the invention and then detecting an increase or decrease in expression (either transcript, translation product, or catalytic product). Some assays are performed with peripheral cells, or other cells, that express an endogenous polypeptide or polynucleotide of the invention.

Polypeptide or polynucleotide expression can be detected in a number of different ways. As described infra, the expression level of a polynucleotide of the invention in a cell can be determined by probing the mRNA expressed in a cell with a probe that specifically hybridizes with a transcript (or complementary nucleic acid derived therefrom) of a polynucleotide of the invention. Probing can be conducted by lysing the cells and conducting Northern blots or without lysing the cells using in situ-hybridization techniques. Alternatively, a polypeptide of the invention can be detected using immunological methods in which a cell lysate is probed with antibodies that specifically bind to a polypeptide of the invention.

Other cell-based assays are reporter assays conducted with cells that do not express a polypeptide or polynucleotide of the invention. Certain of these assays are conducted with a heterologous nucleic acid construct that includes a promoter of a polynucleotide of the invention that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, chloramphenicol acetyl transferase (CAT); Alton and Vapnek (1979) Nature 282:864-869), luciferase, β-galactosidase, green fluorescent protein (GFP) and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. A test compound that either activates the promoter by binding to it or triggers a cascade that produces a molecule that activates the promoter causes expression of the detectable reporter. Certain other reporter assays are conducted with cells that harbor a heterologous construct that includes a transcriptional control element that activates expression of a polynucleotide of the invention and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

The level of expression or activity can be compared to a baseline value. As indicated above, the baseline value can be a value for a control sample or a statistical value that is representative of expression levels for a control population (e.g., healthy individuals). Expression levels can also be determined for cells that do not express a polynucleotide of the invention as a negative control. Such cells generally are otherwise substantially genetically the same as the test cells.

A variety of different types of cells can be utilized in the reporter assays. Cells that may express an endogenous polypeptide or polynucleotide of the invention include, e.g., fibroblast. Cells that do not endogenously express polynucleotides of the invention can be prokaryotic, but are preferably eukaryotic. The eukaryotic cells can be any of the cells typically utilized in generating cells that harbor recombinant nucleic acid constructs. Exemplary eukaryotic cells include, but are not limited to, yeast, and various higher eukaryotic cells such as the COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

### Catalytic Activity

Catalytic activity of polypeptides of the invention can be determined by measuring the production of enzymatic products or by measuring the consumption of substrates. Activity refers to either the rate of catalysis or the ability to the polypeptide to bind (Kₘ) the substrate or release the catalytic product (K_{d}).

Analysis of the activity of polypeptides of the invention are performed according to general biochemical analyses. Such assays include cell-based assays as well as in vitro assays involving purified or partially purified polypeptides or crude cell lysates. The assays generally involve providing a known quantity of substrate and quantifying product as a function of time.

### Validation

Agents that are initially identified by any of the foregoing screening methods can be further tested to validate the apparent activity. Preferably such studies are conducted with suitable animal models. The basic format of such methods involves administering a lead compound identified during an initial screen to an animal that serves as a model for humans and then determining if expression or activity of a polynucleotide or polypeptide of the invention is in fact upregulated. The animal models utilized in validation studies generally are mammals of any kind. Specific examples of suitable animals include, but are not limited to, primates, mice, and rats. As described herein, models using administration of known therapeutics can be useful.

### Animal Models

Animal models also find use in screening for modulators. In one embodiment, invertebrate models such as Drosophila models can be used, screening for modulators of Drosophila orthologs of the human genes disclosed herein. In another embodiment, transgenic animal technology including gene knockout technology, for example as a result of homologous recombination with an appropriate gene targeting vector, or gene overexpression, will result in the absence, decreased or increased expression of a polynucleotide or polypeptide of the invention. The same technology can also be applied to make knockout cells. When desired, tissue-specific expression or knockout of a polynucleotide or polypeptide of the invention may be necessary.

Knockout cells and transgenic mice can be made by insertion of a marker gene or other heterologous gene into an endogenous gene site in the mouse genome via homologous recombination. Such mice can also be made by substituting an endogenous polynucleotide of the invention with a mutated version of the polynucleotide, or by mutating an endogenous polynucleotide, e.g., by exposure to carcinogens.

For development of appropriate stem cells, a DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

B. Modulators of Polypeptides or Polynucleotides of the Invention

The agents tested as modulators of the polypeptides or polynucleotides of the invention can be any small chemical compound, or a biological entity, such as a protein, sugar, nucleic acid or lipid. Alternatively, modulators can be genetically altered versions of a polypeptide or polynucleotide of the invention. Typically, test compounds will be small chemical molecules and peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like. Modulators also include agents designed to reduce the level of mRNA of the invention (e.g. antisense molecules, ribozymes, DNAzymes and the like) or the level of translation from an MRNA.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md., etc.).

C. Solid State and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds are possible using the integrated systems of the invention. More recently, microfluidic approaches to reagent manipulation have been developed.

The molecule of interest can be bound to the solid state component, directly or indirectly, via covalent or non-covalent linkage, e.g., via a tag. The tag can be any of a variety of components. In general, a molecule that binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs, such as agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-Gly sequences of between about 5 and 200 amino acids. Such flexible linkers are known to those of skill in the art. For example, poly(ethelyne glycol) linkers are available from Shearwater Polymers, Inc., Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (see, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank and Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

The invention provides in vitro assays for identifying, in a high throughput format, compounds that can modulate the expression or activity of the polynucleotides or polypeptides of the invention. In a preferred embodiment, the methods of the invention include such a control reaction. For each of the assay formats described, “no modulator” control reactions that do not include a modulator provide a background level of binding activity.

In some assays it will be desirable to have positive controls to ensure that the components of the assays are working properly. At least two types of positive controls are appropriate. First, a known activator of a polynucleotide or polypeptide of the invention can be incubated with one sample of the assay, and the resulting increase in signal resulting from an increased expression level or activity of polynucleotide or polypeptide determined according to the methods herein. Second, a known inhibitor of a polynucleotide or polypeptide of the invention can be added, and the resulting decrease in signal for the expression or activity can be similarly detected.

6. Administration and Pharmaceutical Compositions

Modulators of the polynucleotides or polypeptides of the invention can be administered directly to a mammalian subject for modulation of activity of those molecules in vivo. Administration is by any of the routes normally used for introducing a modulator compound into ultimate contact with the tissue to be treated and is well known to those of skill in the art. Although more than one route can be used to administer a particular composition, a particular route can often provide a more immediate and more effective reaction than another route.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

The modulators (e.g., agonists or antagonists) of the expression or activity of the a polypeptide or polynucleotide of the invention, alone or in combination with other suitable components, can be made into aerosol formulations (i.e., they can be “nebulized”) to be administered via inhalation or in compositions useful for injection. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to effect a beneficial response in the subject over time. The optimal dose level for any patient will depend on a variety of factors including the efficacy of the specific modulator employed, the age, body weight, physical activity, and diet of the patient, on a possible combination with other drugs, and on the severity of the disorder. The size of the dose also will be determined by the existence, nature, and extent of any adverse side effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side effects of the modulator at various concentrations, as applied to the mass and overall health of the subject. Administration can be accomplished via single or divided doses.

### 7. Gene Therapy Applications

A variety of human diseases and conditions such as wound healing can be treated by therapeutic approaches that involve stably introducing a gene into a human cell such that the gene is transcribed and the gene product is produced in the cell. Diseases amenable to treatment by this approach include inherited diseases, including those in which the defect is in a single or multiple genes. Gene therapy is also useful for treatment of acquired diseases and other conditions. For discussions on the application of gene therapy towards the treatment of genetic as well as acquired diseases, see, Miller, Nature 357:455-460 (1992); and Mulligan, Science 260:926-932 (1993). In the context of the present invention, gene therapy can be used for treating a variety of disorders and/or diseases in which the polynucleotides and polypeptides of the invention has been implicated.

A. Vectors for Gene Delivery

For delivery to a cell or organism, the polynucleotides of the invention can be incorporated into a vector. Examples of vectors used for such purposes include expression plasmids capable of directing the expression of the nucleic acids in the target cell. In other instances, the vector is a viral vector system wherein the nucleic acids are incorporated into a viral genome that is capable of transfecting the target cell. In a preferred embodiment, the polynucleotides can be operably linked to expression and control sequences that can direct expression of the gene in the desired target host cells. Thus, one can achieve expression of the nucleic acid under appropriate conditions in the target cell.

B. Gene Delivery Systems

Viral vector systems useful in the expression of the nucleic acids include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the genes of interest are inserted into such vectors to allow packaging of the gene construct, typically with accompanying viral DNA, followed by infection of a sensitive host cell and expression of the gene of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, molecular conjugates of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors are also useful for introducing the nucleic acids of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis-acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide of the invention and thus restore the cells to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

In some embodiments of the invention, an antisense polynucleotide is administered which hybridizes to a gene encoding a polypeptide of the invention. The antisense polypeptide can be provided as an antisense oligonucleotide (see, e.g., Murayama et al., Antisense Nucleic Acid Drug Dev. 7:109-114 (1997)). Genes encoding an antisense nucleic acid can also be provided; such genes can be introduced into cells by methods known to those of skill in the art. For example, one can introduce an antisense nucleotide sequence in a viral vector, such as, for example, in hepatitis B virus (see, e.g., Ji et al., J. Viral Hepat. 4:167-173 (1997)), in adeno-associated virus (see, e.g., Xiao et al., Brain Res. 756:76-83 (1997)), or in other systems including, but not limited, to an HVJ (Sendai virus)-liposome gene delivery system (see, e.g., Kaneda et al., Ann. NY Acad. Sci. 811:299-308 (1997)), a “peptide vector” (see, e.g., Vidal et al., CR Acad. Sci III 32:279-287 (1997)), as a gene in an episomal or plasmid vector (see, e.g., Cooper et al., Proc. Natl. Acad. Sci. U.S.A. 94:6450-6455 (1997), Yew et al. Hum Gene Ther. 8:575-584 (1997)), as a gene in a peptide-DNA aggregate (see, e.g., Niidome et al., J. Biol. Chem. 272:15307-15312 (1997)), as “naked DNA” (see, e.g., U.S. Pat. Nos. 5,580,859 and 5,589,466), in lipidic vector systems (see, e.g., Lee et al., Crit Rev Ther Drug Carrier Syst. 14:173-206 (1997)), polymer coated liposomes (U.S. Pat. Nos. 5,213,804 and 5,013,556), cationic liposomes (Epand et al., U.S. Pat. Nos. 5,283,185; 5,578,475; 5,279,833; and 5,334,761), gas filled microspheres (U.S. Pat. No. 5,542,935), ligand-targeted encapsulated macromolecules (U.S. Pat. Nos. 5,108,921; 5,521,291; 5,554,386; and 5,166,320).

In another embodiment, conditional expression systems, such as those typified by the tet-regulated systems and the RU-486 system, can be used (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These systems impart small molecule control on the expression of the target gene(s) of interest.

In another embodiment, stem cells engineered to express a transcript of interest can implanted into the brain.

C. Pharmaceutical Formulations

When used for pharmaceutical purposes, the vectors used for gene therapy are formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer, or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans; antioxidants, such as ascorbic acid or glutathione; chelating agents; low molecular weight proteins or other stabilizers or excipients. Other physiologically acceptable compounds include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers, or adjuvants can be found in Remington's Pharmaceutical Sciences, Mack Publishing Company, Philadelphia, Pa., 17th ed. (1985).

D. Administration of Formulations

The formulations of the invention can be delivered to any tissue or organ using any delivery method known to the ordinarily skilled artisan. In some embodiments of the invention, the nucleic acids of the invention are formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701.

E. Methods of Treatment

The gene therapy formulations of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced into cells by such methods as microinjection, calcium phosphate precipitation, liposome fusion, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23 (1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

### EXAMPLES

The present invention is more particularly described in the following examples which are intended as illustrative only since numerous modifications and variations therein will be apparent to those skilled in the art. The following examples are intended to illustrate but not limit the invention.

### Example 1

### Wit3.0 Increases the Rate of Wound Closure

Wit3.0 was first characterized from an oral wound, while its expression is reduced in the skin. There are different methods of stimulating the expression and activity of wit3.0 including therapeutic gene transfer, wit3.0 nano-capsule delivery, and/or wit3.0 gene activation.

FIG. 2 shows a mouse skin model, whereby lesions on contra sides of the inferior side of a mouse have been created. A therapeutically effective amount of wit3.0 in buffer (e.g. TE) was administered to the lesion site (FIG. 2, left hand side of the photograph, or right side of the inferior side of the mouse). The wit3.0 treated wound heals faster as indicated by the wound closure (FIG. 2, day 4). Further, the wit3.0 wound closure does not pull apart (stronger) and has reduced scar tissue (FIG. 2, day 7).

### Example 2

### Wit3.0 Decreases the Area of Granulation Formulation

Wit3.0 treated wounds close in a linear fashion. In a typical wound site post tooth-extraction, the placement of the suture decreases the wound area by pulling the wound margins closer together. However, in order to avoid bacterial infection around the suture materials and wound site, sutures are removed within 7 days after the tooth-extraction or injury. Yet, wound healing is often incomplete after one week and the wound is susceptible to reopening. Hence, a method of treating wound closure without reliance on sutures is advantageous.

FIG. 3 shows Confocal Laser Scanning Microscopic photographs of histological evaluation of mouse skin wounds. The tissue collagen is stained with Sirius Red (red filaments). FIG. 3A is a no-treatment wound (control) showing a sharp demarcation (arrows) between the wound edge and the granulation tissue (*). Other hallmarks of skin tissue are evident, including hair follicles and sweat glands (arrowheads). The wound granulation tissue in the untreated control contains immature collagen fibers and are faintly stained with the Sirius Red dye. FIG. 3B is a wit3.0 treated wound showing pulling of the wound margins (double-head arrow) flanked by a small granulation tissue area (*). In contrast to the no-treatment control, the wit3.0 treated control shows the wound margins being pulled closer together and thick and ordered collagen fibers appearing to unite at the bottom of the wound. The proximity of the wound margins to each other and the organization of the collagen fibrils suggest tissue regeneration.

The above results show that the control (FIG. 3A) wound induced a large granulation tissue region, with faintly stained collagen fibers, which are different from surrounding normal skin. In contrast, the wit3.0 treated wound (FIG. 3B) did not induce granulation tissue to the extent as observed in the un-treated, or control. In the treated wound, the wound edges are pulled closer together and in some areas the native collagen fibers start to regenerate.

### Example 3

### Wit3.0 is a Minimally Invasive (MI) Treatment for Periodontitis

Periodontitis is a common gum disease, causing more tooth loss than tooth decay in the human adult population, as well as animals. Currently, the treatment of periodontitis is limited to tooth extraction or surgical removal of inflamed gum tissue. An alternative and non invasive treatment of periodontitis. FIG. 4 shows an experiment whereby periodontitis in rats was created (induced) by performing a suture ligature around the rat molar (FIG. 4, middle and right panel) using a 6.0 suture. After 10 days, inflamed gingival tissue consistent with periodontitis is established (FIG. 4, middle panel). The height of the inflamed gingival tissue in the induced-periodontitis tissue is maintained (double-head arrow), while the underlying alveolar bone is resorbed, leading to the deepened gingival pocket between the tooth and the gums. In contrast to the induced-periodontitis wound, the gingival tissue of the uninduced-periodontitis molar (CON) is firmly attached to the molar teeth. Treatment of the induced-periodontitis with a naked plasmid DNA vector containing the coding sequence of wit3.0 is applied into the gingival pocket (FIG. 4, right panel). After seven days, a significant tightening of the gingival tissue is observed, and the diseased gingival tissue is firmly re-attached to the molar tooth. Thus, direct administration of a therapeutically effective amount of wit3.0 ameliorates periodontitis without a need for surgery or other more evasive treatments.

### Example 4

### Wit3.0 Single Nucleotide Polymorphism (SNP) for Diagnostic Screening of Gum Disease

Single nucleotide polymorphisms (SNP) are small genetic changes, or variations, that can occur within a DNA sequence. SNPs typically occur outside of the gene coding sequences, but those SNPs occurring within a coding sequence are of particular interest because they are more likely to alter or modify the biological function of that protein. Thus, genes associated with a specific disease and their SNPs therein are tools for discovery and detection of disease, facilitating the diagnosis and the treatment of disease.

Wit3.0 has been mapped to chromosome 12p11.23 in human and 4q44 in rat (FIGS. 5A, B and C). Many wit3.0 variants or SNPs have been described and deposited into the NCBI on the world wide web. Out of 40 SNPs found in the wit3.0 allele, two SNPs are located in the coding sequence of exon 5. The SNP in exon 5 changes the deduced amino acid sequence, and results in amino-acid substitution mutations. Thus, four possible human variations of wit3.0 coding sequence exist: GAAGCA=154Glu-155Asp (wild type); GACGCA=154Ala-155Asp; GAACCA=154Glu-155Pro; and GACCCA=154Ala-155Pro. Hence, human wit3.0 genotype may have up to 9 possible combinations of haplotypes.

Methods of wit3.0 SNP genotyping may confer susceptibility or resistance to a disease and determine the severity or progression of disease before the onset of the disease. The characterization of a SNP in a gene particularly known to be associated with a disease trait will elucidate the role of genetic and non-genetic factors. For example, characterization of a SNP in a gene associated with gum disease will assist in the diagnosis and prognosis of treatment for that gum disease (e.g. tooth extraction wound healing, and/or disease following residual ridge alveolar bone resorption).

In one embodiment of the invention, blood samples are collected from a group of individuals affected by the disease and their DNA is analyzed for specific SNPs, or SNP patterns. These SNP patterns are compared to another (control) group who are unaffected by the disease. Detectable differences between the SNP patterns of the experimental (disease) group versus the control (un-diseased) group may be indicative of genetic factor associated with the disease-causing gene. Thus, over time, SNP profiles characteristic of a particular disease, for example, gum disease, is established. Ultimately, healthy patients can be screened for susceptibility to a disease by analysis of their SNP patterns (e.g. wit3.0 SNP patterns).

In another embodiment of the invention, a detectable probe spanning the wit3.0 variant or SNP can be used to selectively hybridize to a target sequence in a sample. Detection and characterization of the particular SNP pattern of the target sequence will assist in the treatment of the disease. With regards to gum disease, there are at least two important variations for residual alveolar bone resorption: short-term and long-term. These variations may show expression of different wit3.0 SNP patterns. Characterizing what types of disease tissue contain which wit3.0 SNP patterns will ultimately support a treatment of the disease (e.g. either conventional or implant-assisted dental prostheses).

Although the present process has been described with reference to specific details of certain embodiments thereof in the above examples, it will be understood that modifications and variations are encompassed within the spirit and scope of the invention. All references cited herein are incorporated by reference in their entirety.

What is claimed is:

1. A method for providing a diagnosis or prognosis for wound healing in a subject, the method comprising the step of identifying one or more single nucleotide polymorphisms in a nucleic acid sample, wherein the single nucleotide polymorphism is indicative of a wound healing response, and wherein at least one of the single nucleotide polymorphisms is selected from the group consisting of nucleotide 739 of SEQ ID NO: 1, nucleotide 740 of SEQ ID NO:1, or nucleotides 739 and 740 of SEQ ID NO:1, thereby providing a prognosis for wound healing of the subject.

2. The method of claim 1, wherein the wound is an oral wound.

3. The method of claim 1, wherein the wound is an oral wound caused by tooth extraction, gingival periodontitis, or residual alveolar resorption.

4. The method of claim 1, wherein the wound is a skin wound.

5. The method of claim 1, wherein the single nucleotide polymorphism is detected using PCR.

6. The method of claim 1, wherein the subject is a human.

7. The method of claim 1, wherein the subject is a non-human animal.

8. A primer that specifically detects SNP 1, SNP 2, or SNP 1 and 2 of human wit3.0.

9. A kit comprising the primer of claim 8.

10. An antibody that specifically detects SNP 1, SNP 2, or SNP 1 and 2 of human wit3.0.

11. A kit comprising the antibody of claim 10.

12. A method of improving wound healing in a subject, the method comprising the step of administering to the subject, at the site of the wound, a therapeutically effective amount of a composition comprising a physiologically acceptable carrier and a nucleic acid molecule comprising a nucleotide sequence encoding an amino acid sequence comprises at least 90% identity to wound inducible transcript-3.0 (wit3.0) (SEQ ID NO:2), wherein the nucleic acid molecule is operably linked to a promoter and expressed such that wound healing is improved.

13. The method of claim 12, wherein the amino acid sequence comprises at least 95% identity to wound inducible transcript-3.0 (wit3.0) (SEQ ID NO:2).

14. The method of claim 12, wherein the amino acid sequence comprises the sequence of SEQ ID NO:2.

15. The method of claim 12, wherein the composition is administered to a fibroblast or a fibroblast-like cell.

16. The method of claim 12, wherein the wound is a skin wound.

17. The method of claim 12, wherein the wound is an oral wound.

18. The method of claim 12, wherein the wound healing is associated with a gum or periodontal disease including, gingival periodontitis, or residual alveolar resorption.

19. The method of claim 12, wherein the nucleic acid molecule is a viral vector.

20. The method of claim 19, wherein the viral vector is a DNA vector.

21. The method of claim 20, wherein the DNA vector is an adenovirus (AV), adeno-associated viral (AAV), or lentiviral vector.

22. The method of claim 12, wherein the nucleic acid molecule is a naked nucleic acid molecule.

23. The method of claim 12, wherein the subject is a non-human animal.

24. The method of claim 12, wherein the subject is a human.


SEQUENCE LISTING (9 sequences)

SEQ ID NO: 1 - DNA, 2797 bp, Homo sapiens
  human wild type wound inducible transcript-3.0 (wit3.0) beta, FGFR1 oncogene partner 2 (FGFR1OP2)
aagcgggtgcgggtcgcggaggttctggtccgcccgggatggggccgagacgagtgggtttcccgtacgtcccgccgctgtgtgagcaggactgtgcgtccgctcgctgagcaggcgcagcgagggcgcggagaccactccgtgggcactccacctcgcgggtggtcgtcgccccagggggactgaagcgcagtggaataactacacgcgtgggcgagcctctgagccgggcttcctctctccgtgctctccctgaggtcctgccgggaactgagaaatgagctgcaccattgagaaggcacttgctgacgctaaagcccttgttgaaaggttgagagatcatgatgatgcagcagagtctctcatcgagcagaccactgccctcagcaagcgagtggaagcgatgaagcagtatcaggaggaaatccaagaacttaatgaagtagcaagacatcggccacgatccacactagttatgggaatccagcaagaaaacagacaaatcagagaattacaacaagaaaacaaagaactgcggacatccctggaagagcaccagtctgccctggaactgataatgagcaagtaccgagagcagatgttcagactgctcatggccagcaagaaagatgacccaggtataataatgaagttaaaagagcaacactcaaagattgacatggtacatcgtaacagctgcgaaggattcttcctggatgcatctcggcacatccttgaagcacctcagcacggactggagaggaggcacttggaggcaaatcagaatgagttgcaagcacatgttgaccaaatcaccgagatggcagcagtaatgagaaaagctattgaaattgacgagcagcagggttgcaaggaacaggagcggatatttcaacttgaacaagaaaataaaggcttgagagagatccttcagataactcgagaatcctttttgaaccttcggaaggatgatgcgtcagagagtacgtctctatctgccttagtgactaacagtgacctgagtctgaggaagagctgagtggctttctgtgcggtcactagaatgggcccagaagtgagtggatgagtgaacataaagcccaacttcagtcagccttttccgtctggtatgtacagggacactggcaaagagacggcagcagaatgtataaccagtggtcataaactagatcccagtcacagtcagaaaggagacgctgagccacagagtgaacagcgagtcttcaaggtgctgctgagcactgcagtttcagaagtgctgttggcttcttagatgctacatagatcaccctgaatactcctaataaatgttggaaagcctaaattatcacaattttaaaagtaaggttggtgtggttttctgaatggaagactttgttctaagtcttaccttcctgttcagtgtcagagttggcatagcttgtgtacataaagctgctgagttggtgctcacctgcccatttgctcggccctcatcaaaggcagttcatgtttaaagactataggaatacgtcacttgcatcacaaacagaagggacatacttacacctaattcccataactcttagctagcagttattcatgactcgcccaagtgtctgcatctcaggaaagaaagacaaagaacatactttctaagaaagacagccatatatagacacattcagtaggttgaactactttgaaagattatgttttggttctctgtnaacaatgaacctggttcttcctcctctattcatcatgtttgaggatacaagacagttcaccaatagacaagagttgacaatttaatgtaatgagataggtatggtcatttccaagttaatttggaattcctttattgttatttaagaaattactcttcatacacttacaaagtagatacaggttccaaaaatcaaagttctgagtctgagtctaagcctcatctagttcttataacctgtgaggtttttcctacaacctgtgactcagaaactggcagtgaagtgtggacacaaagggattgtgctttgcactttaaaaaaaaaaaaagcttcttaaggccacaggatacagaagtgtgctttcataccaactgcaacctaaacttttctgtagtgataagaataaaagattatgagatatggctgaaaatacactatacttgtatctgaagtccatactgaaattagcacaaacagaaagttagacttttcaatatatcatttgttaagtacatattactttgcagtgtaattttatgtgtaatttcatgtattggcaaaattaaaaggacttttcactccagaacctttcattctgaagtttgaggtgagtggggtcataggtcagttaggaaagggccagttccccagatgttaaaccgttctcatgcagaggccttaatattttatatataagtgaatttacttttaagatttttctattgttttttggaaagtaacccttaatttaatggtactttctttcatgtagttttgtcccaggccagaattttaaaaagaagaaaagcggcacagacgagatgagtcagatcgatgtattgagcaggtctattgtgaaacgttatttgagaagtaattatttttataaaaaattatttatcctttgttttcttggatataattttaaggttgtgaatattgaaagacatttgctttgtttttagcaagttttccaccccacccccatccccaaaataaacagctttaagcttacatatgagagtgtgctgaagaggagcctattttgtcaataaagatgagtgttt

SEQ ID NO: 2 - protein, 253 aa, Homo sapiens
  human wild type wound inducible transcript-3.0 (wit3.0) beta, FGFR1 oncogene partner 2 (FGFR1OP2)
MSCTIEKALADAKALVERLRDHDDAAESLIEQTTALSKRVEAMKQYQEEIQELNEVARHRPRSTLVMGIQQENRQIRELQQENKELRTSLEEHQSALELIMSKYREQMFRLLMASKKDDPGIIMKLKEQHSKIDMVHRNSCEGFFLDASRHILEAPQHGLERRHLEANQNELQAHVDQITEMAAVMRKAIEIDEQQGCKEQERIFQLEQENKGLREILQITRESFLNLRKDDASESTSLSALVTNSDLSLRKS

SEQ ID NO: 3 - DNA, 2797 bp, Homo sapiens
  human wound inducible transcript-3.0 (wit3.0) beta, FGFR1 oncogene partner 2 (FGFR1OP2) with SNP 1 and SNP 2 in exon 5
aagcgggtgcgggtcgcggaggttctggtccgcccgggatggggccgagacgagtgggtttcccgtacgtcccgccgctgtgtgagcaggactgtgcgtccgctcgctgagcaggcgcagcgagggcgcggagaccactccgtgggcactccacctcgcgggtggtcgtcgccccagggggactgaagcgcagtggaataactacacgcgtgggcgagcctctgagccgggcttcctctctccgtgctctccctgaggtcctgccgggaactgagaaatgagctgcaccattgagaaggcacttgctgacgctaaagcccttgttgaaaggttgagagatcatgatgatgcagcagagtctctcatcgagcagaccactgccctcagcaagcgagtggaagcgatgaagcagtatcaggaggaaatccaagaacttaatgaagtagcaagacatcggccacgatccacactagttatgggaatccagcaagaaaacagacaaatcagagaattacaacaagaaaacaaagaactgcggacatccctggaagagcaccagtctgccctggaactgataatgagcaagtaccgagagcagatgttcagactgctcatggccagcaagaaagatgacccaggtataataatgaagttaaaagagcaacactcaaagattgacatggtacatcgtaacagctgcgaaggattcttcctggatgcatctcggcacatccttgacccacctcagcacggactggagaggaggcacttggaggcaaatcagaatgagttgcaagcacatgttgaccaaatcaccgagatggcagcagtaatgagaaaagctattgaaattgacgagcagcagggttgcaaggaacaggagcggatatttcaacttgaacaagaaaataaaggcttgagagagatccttcagataactcgagaatcctttttgaaccttcggaaggatgatgcgtcagagagtacgtctctatctgccttagtgactaacagtgacctgagtctgaggaagagctgagtggctttctgtgcggtcactagaatgggcccagaagtgagtggatgagtgaacataaagcccaacttcagtcagccttttccgtctggtatgtacagggacactggcaaagagacggcagcagaatgtataaccagtggtcataaactagatcccagtcacagtcagaaaggagacgctgagccacagagtgaacagcgagtcttcaaggtgctgctgagcactgcagtttcagaagtgctgttggcttcttagatgctacatagatcaccctgaatactcctaataaatgttggaaagcctaaattatcacaattttaaaagtaaggttggtgtggttttctgaatggaagactttgttctaagtcttaccttcctgttcagtgtcagagttggcatagcttgtgtacataaagctgctgagttggtgctcacctgcccatttgctcggccctcatcaaaggcagttcatgtttaaagactataggaatacgtcacttgcatcacaaacagaagggacatacttacacctaattcccataactcttagctagcagttattcatgactcgcccaagtgtctgcatctcaggaaagaaagacaaagaacatactttctaagaaagacagccatatatagacacattcagtaggttgaactactttgaaagattatgttttggttctctgtnaacaatgaacctggttcttcctcctctattcatcatgtttgaggatacaagacagttcaccaatagacaagagttgacaatttaatgtaatgagataggtatggtcatttccaagttaatttggaattcctttattgttatttaagaaattactcttcatacacttacaaagtagatacaggttccaaaaatcaaagttctgagtctgagtctaagcctcatctagttcttataacctgtgaggtttttcctacaacctgtgactcagaaactggcagtgaagtgtggacacaaagggattgtgctttgcactttaaaaaaaaaaaaagcttcttaaggccacaggatacagaagtgtgctttcataccaactgcaacctaaacttttctgtagtgataagaataaaagattatgagatatggctgaaaatacactatacttgtatctgaagtccatactgaaattagcacaaacagaaagttagacttttcaatatatcatttgttaagtacatattactttgcagtgtaattttatgtgtaatttcatgtattggcaaaattaaaaggacttttcactccagaacctttcattctgaagtttgaggtgagtggggtcataggtcagttaggaaagggccagttccccagatgttaaaccgttctcatgcagaggccttaatattttatatataagtgaatttacttttaagatttttctattgttttttggaaagtaacccttaatttaatggtactttctttcatgtagttttgtcccaggccagaattttaaaaagaagaaaagcggcacagacgagatgagtcagatcgatgtattgagcaggtctattgtgaaacgttatttgagaagtaattatttttataaaaaattatttatcctttgttttcttggatataattttaaggttgtgaatattgaaagacatttgctttgtttttagcaagttttccaccccacccccatccccaaaataaacagctttaagcttacatatgagagtgtgctgaagaggagcctattttgtcaataaagatgagtgttt

SEQ ID NO: 4 - protein, 253 aa, Homo sapiens
  human wound inducible transcript-3.0 (wit3.0) beta, FGFR1 oncogene partner 2 (FGFR1OP2) with SNP 1 and SNP 2 haplotypes
MSCTIEKALADAKALVERLRDHDDAAESLIEQTTALSKRVEAMKQYQEEIQELNEVARHRPRSTLVMGIQQENRQIRELQQENKELRTSLEEHQSALELIMSKYREQMFRLLMASKKDDPGIIMKLKEQHSKIDMVHRNSCEGFFLDASRHILDPPQHGLERRHLEANQNELQAHVDQITEMAAVMRKAIEIDEQQGCKEQERIFQLEQENKGLREILQITRESFLNLRKDDASESTSLSALVTNSDLSLRKS

SEQ ID NO: 5 - DNA, 12 bp, Artificial Sequence
  Description of Artificial Sequencehuman wound inducible transcript-3.0 (wit3.0) beta, FGFR1 oncogene partner 2 (FGFR1OP2) wild type haplotype
cttgaagcacct

SEQ ID NO: 6 - DNA, 12 bp, Artificial Sequence
  Description of Artificial Sequencehuman wound inducible transcript-3.0 (wit3.0) beta, FGFR1 oncogene partner 2 (FGFR1OP2) SNP1 haplotype
cttgacgcacct

SEQ ID NO: 7 - DNA, 12 bp, Artificial Sequence
  Description of Artificial Sequencehuman wound inducible transcript-3.0 (wit3.0) beta, FGFR1 oncogene partner 2 (FGFR1OP2) SNP2  haplotype
cttgaaccacct

SEQ ID NO: 8 - DNA, 12 bp, Artificial Sequence
  Description of Artificial Sequencehuman wound inducible transcript-3.0 (wit3.0) beta, FGFR1 oncogene partner 2 (FGFR1OP2) SNP1 and SNP2 haplotype
cttgacccacct

SEQ ID NO: 9 - protein, 200 aa, Artificial Sequence
  Description of Artificial Sequencepoly-Gly flexible linker, tag
GGGGGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX